(19) United States
(12) Patent Application Publication
(10) Pub. No.: US 2015/0328179 A1
(43) Pub. Date: Nov. 19, 2015

(54) DICAFFEOYLQUINIC ACID-CONTAINING DRINK

(71) Applicant: KAO CORPORATION, Chuo-ku, Tokyo (JP)

(72) Inventors: Yu NAKASHIMA, Sumida-ku (JP); Yasushi SHIOYA, Sumida-ku (JP)

(73) Assignee: KAO CORPORATION, Chuo-ku, Tokyo (JP)

(21) Appl. No.: 14/758,107

(22) PCT Filed: Dec. 26, 2013
(86) PCT No.: PCT/JP2013/084988

§ 371 Date: Jun. 26, 2015

(30) Foreign Application Priority Data
Dec. 28, 2012 (JP) 2012-289015

(51) Int. Cl. A61K 31/235; A23L 1/30; A23L 1/305; A61K 31/198; A23L 2/52

(52) U.S. Cl. CPC A61K 31/235; A61K 31/198; A23L 2/52; A23L 1/3051; A23L 1/3002; A23V 2002/00

(57) ABSTRACT

A dicaffeoylquinic acid-containing beverage of the present invention includes the following components (A) and (B): (A) 0.02 to 0.18 mass % of dicaffeoylquinic acids; and (B) 0.1 to 1.0 mass % of L-arginine, in which a mass ratio between the component (A) and the component (B), [(B)/(A)], is from 2 to 18.

### FIELD OF THE INVENTION

The present invention relates to a dicaffeoylquinic acid-containing beverage.

### BACKGROUND OF THE INVENTION

In recent years, with increasing health consciousness, physiological actions of chlorogenic acids, such as an antioxidant action and a blood-pressure-lowering action, have attracted attention (Patent Document 1). A typical example of a beverage containing the chlorogenic acids is a roasted coffee beverage. The roasted coffee beverage has a good aroma, body, adequate astringency, and the like originating from roasted coffee beans, and has high preference. However, the beverage has strong bitterness, which may cause a trouble in continuous ingestion.

Then, as a technology for reducing the bitterness of the roasted coffee beverage, there has been proposed, for example, a method involving adding one or more of amino acids selected from the group consisting of L-ornithine hydrochloride, D,L-alanine, L-valine, and glycine (Patent Document 2).

In addition, it has been reported that isochlorogenic acid has an aftertaste including astringent taste like a metal, and the aftertaste is improved by oligosaccharides mainly containing mannose (Patent Document 3).

Meanwhile, it has been reported that a basic amino acid and an acidic amino acid can suppress turbidity of an acidic beverage containing an extract of green coffee beans (Patent Document 4).

### CITATION LIST

### Patent Document

[Patent Document 1] JP-A-2002-87977

[Patent Document 2] JP-A-2010-148453

[Patent Document 3] JP-A-2009-165498

[Patent Document 4] JP-A-2012-110322

### Non Patent Document

[Non Patent Document 1] Molecular Pharmacology October 1996 Vol. 50 no. 4 P. 846-855

[Non Patent Document 2] Kaffee and Tee Markt, 1982, 32(21), P. 3-6

### SUMMARY OF THE INVENTION

The present invention provides a dicaffeoylquinic acid-containing beverage, including the following components (A) and (B) : (A) 0.02 to 0.18 mass % of dicaffeoylquinic acids; and (B) 0.1 to 1.0 mass % of L-arginine, in which a mass ratio between the component (A) and the component (B), [(B)/(A)], is from 2 to 18.

The present invention provides an astringency-suppressing agent for dicaffeoylquinic acids, including L-arginine as an active ingredient.

Also, the present invention provides an astringency-suppressing method for dicaffeoylquinic acids, including blending L-arginine in a composition containing dicaffeoylquinic acids.

Further, the present invention provides a use of L-arginine for suppressing astringency of dicaffeoylquinic acids.

### DETAILED DESCRIPTION OF THE INVENTION

The physiological effects of chlorogenic acids are known to be enhanced by dicaffeoylquinic acids in the chlorogenic acids (Non Patent Document 1). The dicaffeoylquinic acids are contained in green coffee beans at a high concentration, but are decomposed by roasting the green coffee beans (Non Patent Document 2). Therefore, in order to more effectively exert the physiological effects of the chlorogenic acids, it is advantageous to use an extract of green coffee beans.

Then, the inventor of the present invention made investigations to develop a beverage containing a high concentration of dicaffeoylquinic acids by use of an extract of green coffee beans, and as a result, found that the beverage provides a new problem, that is, strong astringency originating from dicaffeoylquinic acids, which is not caused by an extract of roasted coffee beans.

The present invention relates to a dicaffeoylquinic acid-containing beverage containing a high concentration of dicaffeoylquinic acids and having suppressed astringency.

The inventor of the present invention found that the problem can be solved by incorporating a specific amino acid in dicaffeoylquinic acids and controlling each of the concentrations of the dicaffeoylquinic acids and specific amino acid and the content ratio of the specific amino acid to the dicaffeoylquinic acids in a beverage within specific ranges.

According to the present invention, it is possible to provide a dicaffeoylquinic acid-containing beverage having suppressed astringency. The dicaffeoylquinic acid-containing beverage of the present invention contains a high concentration of dicaffeoylquinic acids effective for exertion of physiological actions, has good taste and flavor, and can be taken continuously. Therefore, the beverage can be expected to have satisfactory physiological actions provided by the dicaffeoylquinic acids.

According to the present invention, it is also possible to provide an astringency-suppressing agent for dicaffeoylquinic acids and an astringency-suppressing method for dicaffeoylquinic acids.

The dicaffeoylquinic acid-containing beverage of the present invention contains dicaffeoylquinic acids (A) at as high a concentration as from 0.02 to 0.18 mass %. From the viewpoint of astringency, the concentration is preferably 0.15 mass % or less, more preferably 0.12 mass % or less, even more preferably 0.10 mass % or less, and from the viewpoint of physiological activity, the concentration is preferably 0.03 mass % or more, more preferably 0.04 mass % or more, even more preferably 0.05 mass % or more. From the viewpoints of both astringency and physiological effects, the content of the dicaffeoylquinic acids (A) in the beverage is preferably from 0.03 to 0.15 mass %, more preferably from 0.04 to 0.12 mass %, even more preferably from 0.05 to 0.10 mass %.

The term “dicaffeoylquinic acids” as used herein is a general term collectively encompassing 3,4-dicaffeoylquinic acid, 3,5-dicaffeoylquinic acid, and 4,5-dicaffeoylquinic acid, and in the present invention, at least one out of the three kinds of dicaffeoylquinic acids needs to be incorporated. It should be noted that the content of the dicaffeoylquinic acids is defined based on the total amount of the three of dicaffeoylquinic acids. In addition, the dicaffeoylquinic acids are also referred to as isochlorogenic acids.

The dicaffeoylquinic acid-containing beverage of the present invention may contain polyphenols other than the dicaffeoylquinic acids, and examples thereof may include monocaffeoylquinic acids and monoferuloylquinic acids. As the monocaffeoylquinic acids, there are given, for example, 3-caffeoylquinic acid, 4-caffeoylquinic acid, and 5-caffeoylquinic acid, and as the monoferuloylquinic acids, there are given, for example, 3-feruloylquinic acid, 4-feruloylquinic acid, and 5-feruloylquinic acid. At least one out of the three of monocaffeoylquinic acids needs to be incorporated, and at least one out of the three of monoferuloylquinic acids needs to be incorporated. Herein, the three of dicaffeoylquinic acids, the three of monocaffeoylquinic acids, and the three of monoferuloylquinic acids are collectively referred to as “chlorogenic acids”.

From the viewpoint of physiological actions, the content of monocaffeoylquinic acids and monoferuloylquinic acids in Lhe dicaffeoylquinic acid-containing beverage of the present invention is preferably 0.05 mass % or more, more preferably 0.1 mass % or more, more preferably 0.15 mass % or more, even more preferably 0.2 mass % or more, and from the viewpoint of taste and flavor, the content is preferably 0.6 mass % or less, more preferably 0.5 mass % or less, more preferably 0.4 mass % or less, more preferably 0.35 mass % or less, even more preferably 0.3 mass % or less. The content of monocaffeoylquinic acids and monoferuloylquinic acids in the beverage falls within the range of preferably from 0.05 to 0.6 mass %, more preferably from 0.1 to 0.5 mass %, more preferably from 0.15 to 0.4 mass %, more preferably from 0.2 to 0.35 mass %, even more preferably from 0.2 to 0.3 mass %. It should be noted that the content of monocaffeoylquinic acids and monoferuloylquinic acids is defined based on the total amount of the above-mentioned six of compounds.

From the viewpoint of physiological effects, the mass ratio of the dicaffeoylquinic acids (A) to chlorogenic acids in the dicaffeoylquinic acid-containing beverage of the present invention is preferably 0.07 or more, more preferably 0.08 or more, more preferably 0.1 or more, even more preferably 0.12 or more. The upper limit of the mass ratio of the dicaffeoylquinic, acids (A) to chlorogenic acids is not particularly limited and may be 1.0. From the viewpoint of productivity, the upper limit is preferably 0.9 or less, more preferably 0.8 or less, more preferably 0.6 or less, even more preferably 0.4 or less. From the viewpoints of both physiological effects and productivity, the mass ratio of the dicaffeoylquinic acids (A) to chlorogenic acids in the beverage is preferably from 0.07 to 1.0, more preferably from 0.07 to 0.9, more preferably from 0.08 to 0.8, more preferably from 0.1 to 0.6, even more preferably from 0.12 to 0.4. It should be noted that the contents of “dicaffeoylquinic acids” and “chlorogenic acids” is measured in accordance with “Analysis of chlorogenic acids” described in Examples to be shown later.

The dicaffeoylquinic acid-containing beverage of the present invention contains L-arginine (B) in order to suppress astringency. The content of the L-arginine (B) in the dicaffeoylquinic acid-containing beverage of the present invention is from 0.1 to 1.0 mass %. From the viewpoint of bitterness originating from the L-arginine, the content is preferably 0.8 mass % or less, more preferably 0.6 mass % or less, more preferably 0.5 mass % or less, more preferably 0.48 mass % or less, even more preferably 0. 4 mass % or less, and from the viewpoint of suppressing astringency of the dicaffeoylquinic acids, the content is preferably 0.12 mass % or more, more preferably 0.14 mass % or more, more preferably 0.16 mass % or more, even more preferably 0.18 mass % or more. From the viewpoint of suppressing both bitterness of the L-arginine and astringency of the dicaffeoylquinic acids, the content of the L-arginine (B) in the beverage is preferably from 0.12 to 0.8 mass %, more preferably from 0.14 to 0.6 mass %, more preferably from 0.16 to 0.5 mass %, more preferably from 0.18 to 0.48 mass %, even more preferably from 0.18 to 0.4 mass %. It should be noted that the amount of the L-arginine is measured in accordance with “Analysis of free arginine” described in Examples to be shown later, and the amount of free arginine is regarded as the amount of L-arginine.

In addition, in the present invention, in order to suppress astringency, the content ratio of the L-arginine (B) to the dicaffeoylquinic acids (A) is controlled within a specific range. Specifically, the content mass ratio between the dicaffeoylquinic acids (A) and the L-arginine (B) in the beverage, [(B)/(A)], is from 2 to 18. From the viewpoint of bitterness originating from L-arginine, the content mass ratio is preferably 17 or less, more preferably 16 or less, more preferably 15 or less, even more preferably 10 or less, and from the viewpoint of suppressing astringency of dicaffeoylquinic acids, the content mass ratio is preferably 3 or more, more preferably 4 or more, even more preferably 5 or more. From the viewpoint of suppressing both the bitterness originating from L-arginine and suppressing the astringency of dicaffeoylquinic acids, the mass ratio in the beverage, [(B)/(A)], is preferably from 3 to 17, more preferably from 4 to 16, more preferably from 5 to 15, even more preferably from 5 to 10.

The L-arginine may be a naturally-occurring product or a chemically synthesized product, or may be a commercially available product. An example of the naturally-occurring product is an extract from soft roe of fish such as herring or salmon, and an example of the chemically synthesized product is a product obtained by fermentation. Those products may be purified by column chromatography or the like, if necessary.

The L-arginine to be used in the present invention is preferably a crystal or crystalline powder, and preferably has a purity of 98% or more and a loss on drying of 0.2 mass % or less. It should be noted that the particle size distribution of the L-arginine is not particularly limited. Examples of such commercially available products include L-arginine manufactured by KYOWA HAKKO BIO CO., LTD and PROTEIN CHEMICAL Co., Ltd.

The dicaffeoylquinic acid-containing beverage of the present invention may contain caffeine (C). From the viewpoint of bitterness, the content of the caffeine (C) in the beverage is preferably 0.07 mass % or less, more preferably 0.06 mass % or less, more preferably 0.05 mass % or less, more preferably 0.02 mass % or less, more preferably 0.015 mass % or less, more preferably 0.007 mass % or less, more preferably less than 0.003 mass %, even more preferably 0.002 mass % or less. It should be noted that the I ower limit of the content of the caffeine (C) is not particularly limited, and may be 0. From the viewpoint of productivity, the content of the caffeine (C) is preferably 0.00001 mass % or more, more preferably 0.00005 mass % or more, even more preferably 0.0001 mass % or more. From the viewpoints of both bitterness and productivity, the content of the caffeine in the beverage is preferably from 0.00001 to 0.015 mass %, more preferably from 0.00005 to 0.007 mass %, more preferably 0.00005 mass % or more and less than 0.003 mass %, more preferably from 0.00005 to 0.002 mass %, even more preferably from 0.0001 to 0.002 mass %.

The dicaffeoylquinic acid-containing beverage of the present invention may contain potassium. From the viewpoint of suppressing grassy-smelling, the content of the potassium (D) is preferably from 0.00001 to 0.06 mass %, more preferably from 0.0001 to 0.03 mass %, even more preferably 0.001 to 0.02 mass %. In addition, in order to suppress the grassy-smelling, the content ratio of the potassium (D) to the dicaffeoylquinic acids (A) is preferably a specific value or less. Specifically, the content mass ratio between the dicaffeoylquinic acids (A) and the potassium (D) in the beverage, [(D)/(A)], is preferably 0.5 or less, more preferably 0.4 or less, more preferably 0.3 or less, even more preferably 0.2 or less. It should be noted that the lower limit of the mass ratio may be 0, and from the viewpoint of productivity, the mass ratio [(D)/(A)] is preferably 0.0005 or more, more preferably 0.005 or more, even more preferably 0.015 or more. From the viewpoints of both suppressing grassy-smelling and productivity, the moss ratio in the beverage, [(D)/(A)], is preferably from 0.0005 to 0.5, more preferably from 0.0005 to 0.4, more preferably from 0.005 to 0.3, even more preferably from 0.015 to 0.2.

If necessary, the dicaffeoylquinic acid-containing beverage of the present invention may contain one or two or more of additives such as acidulants, pH adjusters, milk constituents, sweeteners, bitter taste suppressants, antioxidants, flavors, inorganic salts, pigments, emulsifiers, preservatives, seasonings, and quality stabilizers. It should be noted that the blending amount of any such additive may be appropriately set as long as the object of the present invention is not impaired.

The dicaffeoylquinic acid-containing beverage of the present invention can be produced by blending dicaffeoylquinic acids (A) and L-arginine (B) and adjusting the concentrations of the dicaffeoylquinic acids and L-arginine and the mass ratio [(B)/(A)] within the above-mentioned ranges.

In the present invention, commercially available reagents may be used as the dicaffeoylquinic acids (A), and a product collected by fractionation from a plant extract containing the dicaffeoylquinic acids (A) at a high concentration may be used.

The plant extract is not particularly limited as long as it contains dicaffeoylquinic acids, and examples thereof include one or two or more selected from extracts of sunflower seeds, unripe apple, coffee beans, leaves of Simon batatas, cones of Pinaceae plants, seed husks of Pinaceae plants, sugar cane, leaves of Nandina domestica, burdock, the skin of eggplant, the fruit of Japanese apricot, coltsfoot, and Vitaceae plants. Of those, an extract of coffee beans is preferred from the viewpoint of, for example, the content of the dicaffeoylquinic acids.

From the viewpoint of, for example, the content of the dicaffeoylquinic acids, the coffee beans to be used for the extraction are preferably one or two or more selected from the group consisting of green coffee beans and lightly roasted coffee beans. The lightly roasted coffee beans have an L value of preferably 27 or more, more preferably 29 or more, from the viewpoint of the content of the dicaffeoylquinic acids, and have an L value of preferably less than 62, more preferably 60 or less, even more preferably 55 or less, from the viewpoint of taste and flavor. The L value of the lightly roasted coffee beans falls within the range of preferably 27 or more and less than 62, more preferably from 27 to 60, even more preferably from 29 to 55. The term “L value” as used herein refers to a value determined by measuring a lightness value of roasted coffee beans using a colorimeter, if the L value of black is regarded as 0 and the L value of white is regarded as 100, respectively. The extract of green coffee beans may be a commercially available product, and examples thereof include “Flavor holder FH1041” manufactured by Hasegawa Co., Ltd., “Green coffee bean extract P” manufactured by Oryza Oil & Fat Chemical Co., Ltd., and “OXCH100” manufactured by ToyoHakko Co., Ltd.

The kind of coffee beans may be any of Coffee Arabica, Coffee Robusta, Coffee Liberica and Coffee Arabusta. In addition, for example, Brazil, Colombia, Tanzania, Mocha, Kilimanjaro, Mandheling, Blue Mountain, and Guatemala are given as production regions of the coffee beans.

The extraction method and extraction conditions thereof are not particularly limited, and a method disclosed in, for example, JP-A-58-138347, JP-A-59-51763, JP-A-62-111671, and JP-A-5-236918 may be adopted.

In addition, a fractionation method is not particularly limited, and a known method may be employed. For example, various chromatography such as ion chromatography, molecular sieving chromatography, and reversed phase chromatography may be performed singly or in combination in any order.

The dicaffeoylquinic acid-containing beverage of the present invention may be provided by filling a conventional package, such as a molded container formed of polyethylene terephthalate as a main component (a so-called PET bottle), a metal can, a paper package in combination with metal foil or a plastic film, and a bottle, with the beverage.

Further, the dicaffeoylquinic acid-containing beverage can be produced, for example, by putting the beverage in a container such as a metal can and, when sterilized with heating is feasible, conducting heat sterilization under the sterilization conditions defined by the corresponding law (the Food Sanitation Act in Japan). In the case of a container such as a PET bottle or a paper container to which retort sterilization cannot be applied, the dicaffeoylquinic acid-containing beverage is previously sterilized for example, at a high temperature for a short time sterilization under the equivalent conditions as aforementioned above, by a plate-type heat exchanger or the like, is cooled to a predetermined temperature, and then is filling in a container.

In addition, in the astringency-suppressing agent for dicaffeoylquinic acids, astringency-suppressing method for dicaffeoylquinic acids, and use for suppressing astringency of dicaffeoylquinic acids according to the present invention, L-arginine is used as an active ingredient, and theny are also applied not only to dicaffeoylquinic acids but also to a composition containing dicaffeoylquinic acids.

The composition containing dicaffeoylquinic acids is not particularly limited as long as it contains dicaffeoylquinic acids, and examples thereof include a plant extract containing dicaffeoylquinic acids. Examples of such plant extract include one or two or more selected from the group consisting of extracts of sunflower seeds, unripe apple, coffee beans, leaves of Simon batatas, cones of Pinaceae plants, seed husks of Pinaceae plants, sugar cane, leaves of Nandina domestica, burdock, the skin of eggplant, the fruit of Japanese apricot, coltsfoot, and Vitaceae plants. Of those, a coffee extract obtained from coffee beans is preferred from the viewpoint of the content of the dicaffeoylquinic acids.

It should be noted that examples of a form of the composition containing dicaffeoylquinic acids include a liquid, a powder, a granule, and a tablet, and the form may be appropriately selected. When the composition containing dicaffeoylquinic acids is a liquid, the composition maybe usedwithout further treatment, or if necessary, after dilution with water or concentration. Examples of the liquid include an aqueous solution and a hydroalcoholic solution, and the alcohol content in the hydroalcoholic solution may be appropriately selected. In addition, a solid composition containing chlorogenic acids can be obtained by drying a liquid composition containing chlorogenic acids through freeze drying, spray drying, or the like.

In addition, the astringency-suppressing agent, astringency-suppressing method, and use for suppressingastri.ngency of the present invention are preferably applied to a food and beverage containing dicaffeoylquinic acids. Examples of the food and beverage containing dicaffeoylquinic acids include a coffee beverage and a concentrated coffee composition such as a dilutable portion-type beverage or instant coffee. The instant coffee may have a form of a product weighed with a spoon for preparation, a permeable infusion package, or a stick-type product packaged in an amount of a cup of coffee.

It should be noted that specific constituents of the dicaffeoylquinic acids and L-arginine are as described above, and the same constituent as the above-mentioned dicaffeoylquinic acid-containing beverage may be applied to the composition containing dicaffeoylquinic acids.

The present invention further discloses the following beverage, agent, method, and use, regarding the above-mentioned embodiments.

<1-1>

A dicaffeoylquinic acid-containing beverage, comprising the following components (A) and (B):

(A) 0.02 to 0.18 mass % of dicaffeoylquinic acids; and

(B) 0.1 to 1.0 mass % of L-arginine,

in which a content mass ratio between the component (A) and the component (B), [(B)/(A)], is from 2 to 18.
<1-2>

The dicaffeoylquinic acid-containing beverage according to the above-mentioned item <1-1>, in which the content of the dicaffeoylquinic acids (A) is preferably 0.15 mass % or less, more preferably 0.12 mass % or less, even more preferably 0.10 mass % or less, and is preferably 0.03 mass % or more, more preferably 0.04 mass % or more, even more preferably 0.05 mass % or more.

<1-3>

The dicaffeoylquinic acid-containing beverage according to the above-mentioned item <1-1> or <1-2>, in which the content of the dicaffeoylquinic acids (A) is preferably from 0.03 to 0.15 mass %, more preferably from 0.04 to 0.12 mass %, even more preferably from 0.05 to 0.10 mass %.

<1-4>

The dicaffeoylquinic acid-containing beverage according to any one of the above-mentioned items <1-1> to <1-3>, in which the dicaffeoylquinic acids (A) are preferably at least one selected from the group consisting of 3,4-dicaffeoylquinic acid, 3,5-dicaffeoylquinic acid, and 4,5-dicaffeoylquinic acid.

<1-5>

The dicaffeoylquinic acid-containing beverage according to any one of the above-mentioned items <1-1> to <1-4>, preferably further comprising polyphenols other than the dicaffeoylquinic acids (A).

<1-6>

The dicaffeoylquinic acid-containing beverage according to the above-mentioned item <1-5>, in which the polyphenols other than the dicaffeoylquinic acids (A) are preferably at least one selected from the group consisting of monocaffeoylquinic acids and monoferuloylquinic acids, more preferably at least one selected from the group consisting of 3-caffeoylquinic acid, 4-caffeoylquinic acid, 5-caffeoylquinic acid, 3-feruloylquinic acid, 4-feruloylquinic acid, and 5-feruloylquinic acid.

<1-7>

The dicaffeoylquinic acid-containing beverage according to the above-mentioned item <1-6>, in which the content of the monocaffeoylquinic acids and the monoferuloylquinic acids is preferably 0.05 mass % or more, more preferably 0.1 mass % or more, more preferably 0.15 mass % or more, even more preferably 0.2 mass % or more, and is preferably 0.6 mass % or less, more preferably 0.5 mass % or less, more preferably 0.4 mass % or less, more preferably 0.35 mass % or less, even more preferably 0.3 mass % or less.

<1-8>

The dicaffeoylquinic acid-containing beverage according to the above-mentioned item <1-6> or <1-7>, in which the content of the monocaffeoylquinic acids and the monoferuloylquinic acids is preferably from 0.05 to 0.6 mass %, more preferably from 0.1 to 0.5 mass %, more preferably from 0.15 to 0.4 mass %, more preferably from 0.2 to 0.35 mass %, even more preferably from 0.2 to 0.3 mass %.

<1-9>

The dicaffeoylquinic acid-containing beverage according to any one of the above-mentioned items <1-1> to <1-8>, in which the mass ratio of the dicaffeoylquinic acids (A) to chlorogenic acids is preferably 0.07 or more, more preferably 0.08 or more, more preferably 0.1 or more, even more preferably 0.12 or more, and is preferably 1.0 or less, more preferably 0.9 or less, more preferably 0.8 or less, more preferably 0.6 or less, even more preferably 0.4 or less.

<1-10>

The dicaffeoylquinic acid-containing beverage according to any one of the above-mentioned items <1-1> to <1-9>, in which the mass ratio of the dicaffeoylquinic acids (A) to chlorogenic acids is preferably 1.0, more preferably from 0.07 to 1.0, more preferably from 0.07 to 0.9, more preferably from 0.08 to 0.8, more preferably from 0.1 to 0.6, even more preferably from 0.12 to 0.4.

<1-11>

The dicaffeoylquinic acid-containing beverage according to any one of the above-mentioned items <1-1> to <1-10>, in which the content of the L-arginine (B) is preferably 0.8 mass % or less, more preferably 0.6 mass % or less, more preferably 0.5 mass % or less, more preferably 0.48 mass % or less, even more preferably 0.4 mass % or less, and is preferably 0.12 mass % or more, more preferably 0.14 mass % or more, more preferably 0.16 mass % or more, even more preferably 0.18 mass % or more.

<1-12>

The dicaffeoylquinic acid-containing beverage according to any one of the above-mentioned items <1-1> to <1-11>, in which the content of the L-arginine (B) is preferably from 0.12 to 0.8 mass %, more preferably from 0.14 to 0.6 mass %, more preferably from 0.16 to 0.5 mass %, more preferably from 0.18 to 0.48 mass %, even more preferably from 0.18 to 0.4 mass %.

<1-13>

The dicaffeoylquinic acid-containing beverage according to any one of the above-mentioned items <1-1> to <1-12>, in which a content mass ratio between the dicaffeoylquinic acids (A) and the L-arginine (B), [(B)/(A)], is preferably 17 or less, more preferably 16 or less, more preferably 15 or less, even more preferably 10 or less, and is preferably 3 or more, more preferably 4 or more, even more preferably 5 or more.

<1-14>

The dicaffeoylquinic acid-containing beverage according to any one of the above-mentioned items <1-1> to <1-13>, in which the content mass ratio between the dicaffeoylquinic acids (A) and the L-arginine (B), [(B)/(A)], is preferably from 3 to 17, more preferably from 4 to 16, more preferably from 5 to 15, even more preferably from 5 to 10.

<1-15>

The dicaffeoylquinic acid-containing beverage according to any one of the above-mentioned items <1-1> to <1-14>, in which the L-arginine (B) is preferably a crystal or crystalline powder.

<1-16>

The dicaffeoylquinic acid-containing beverage according to any one of the above-mentioned items <1-1> to <1-15>, in which the L-arginine (B) has a purity of 98% or more and a loss on drying of 0.2 mass % or less.

<1-17>

The dicaffeoylquinic acid-containing beverage according to any one of the above-mentioned items <1-1> to <1-16>, preferably further comprising (C) caffeine.

<1-18>

The dicaffeoylquinic acid-containing beverage according to the above-mentioned item <1-17>, in which the content of the caffeine (C) is preferably 0.07 mass % or less, more preferably 0.06 mass % or less, more preferably 0.05 mass % or less, more preferably 0.02 mass % or less, more preferably 0.015 mass % or less, more preferably 0.007 mass % or less, more preferably less than 0.003 mass %, even more preferably 0.002 mass % or less.

<1-19>

The dicaffeoylquinic acid-containing beverage according to the above-mentioned item <1-17> or <1-18>, in which the content of the caffeine (C) is preferably 0 mass % or more, more preferably 0.00001 mass % or more, more preferably 0.00005 mass % or more, even more preferably 0.0001 mass % or more.

<1-20>

The dicaffeoylquinic acid-containing beverage according to any one of the above-mentioned items <1-17> to <1-19>, in which the content of the caffeine (C) is preferably from 0.00001 to 0.015 mass %, more preferably from 0.00005 to 0.007 mass %, more preferably 0.00005 mass % or more and less than 0.003 mass %, more preferably from 0.00005 to 0.002 mass %, even more preferably from 0.0001 to 0.002 mass %, and may be 0 mass %.

<1-21>

The dicaffeoylquinic acid-containing beverage according to any one of the above-mentioned items <1-1> to <1-20>, preferably further comprising potassium (D).

<1-22>

The dicaffeoylquinic acid-containing beverage according to the above-mentioned item <1-21>, in which the content of the potassium (D) is preferably from 0.00001 to 0.06 mass %, more preferably from 0.0001 to 0.03 mass %, even more preferably from 0.001 to 0.02 mass %.

<1-23>

The dicaffeoylquinic acid-containing beverage according to the above-mentioned item <1-21> or <1-22>, in which a content mass ratio between the dicaffeoylquinic acids (A) and the potassium (D), [(D)/(A)], is preferably 0.5 or less, more preferably 0.4 or less, more preferably 0.3 or less, even more preferably 0.2 or less.

<1-24>

The dicaffeoylquinic acid-containing beverage according to any one of the above-mentioned items <1-21> to <1-23>, in which the content mass ratio between the dicaffeoylquinic acids (A) and the potassium (D), [(D)/(A)], is preferably 0.0005 or more, more preferably 0.005 or more, even more preferably 0.015 or more.

<1-25>

The dicaffeoylquinic acid-containing beverage according to any one of the above-mentioned items <1-21> to <1-24>, in which the content mass ratio between the dicaffeoylquinic acids (A) and the potassium (D), [(D)/(A)], is preferably from 0.0005 to 0.5, more preferably from 0.0005 to 0.4, more preferably from 0.005 to 0.3, even more preferably from 0.015 to 0.2, or may be 0.

<1-26>

The dicaffeoylquinic acid-containing beverage according to any one of the above-mentioned items <1-1> to <1-25>, preferably further comprising one or two or more selected from the group consisting of acidulants, pH adjusters, milk constituents, sweeteners, bitter taste suppressants, antioxidants, flavors, inorganic salts, pigments, emulsifiers, preservatives, seasonings, and quality stabilizers.

<1-27>

The dicaffeoylquinic acid-containing beverage according to any one of the above-mentioned items <1-1> to <1-26>, in which the dicaffeoylquinic acids (A) are derived from preferably an extract of one or two or more of plants selected from the group consisting of sunflower seeds, unripe apple, coffee beans, leaves of Simon batatas, cones of Pinaceae plants, seed husks of Pinaceae plants, sugar cane, leaves of Nandina domestica, burdock, the skin of eggplant, the fruit of Japanese apricot, coltsfoot, and Vitaceae plants, more preferably an extract of coffee beans.

<1-28>

The dicaffeoylquinic acid-containing beverage according to the above-mentioned item <1-27>, in which the extract of coffee beans is preferably an extract of one or two or more selected from the group consisting of green coffee beans and lightly roasted coffee beans, more preferably an extract of green coffee beans.

<1-29>

The dicaffeoylquinic acid-containing beverage according to the above-mentioned item <1-28>, in which the lightly roasted coffee beans have an L value of preferably 27 or more, more preferably 29 or more, and of preferably less than 62, more preferably 60 or less, even more preferably 55 or less.

<1-30>

The dicaffeoylquinic acid-containing beverage according Lo the above-mentioned item <1-28> or <1-29>, in which the lightly roasted coffee beans have an L value of preferably 27 or more and less than 62, more preferably from 27 to 60, even more preferably from 29 to 55.

<1-31>

The dicaffeoylquinic acid-containing beverage according to any one of the above-mentioned items <1-1> to <1-30>, in which is preferably a dicaffeoylquinic acid-containing beverage packaged in a container.

<1-32>

The dicaffeoylquinic acid-containing beverage according to the above-mentioned item <1-31>, in which the container is preferably a PET bottle, a metal can, a paper container combined with a metal foil or a plastic film, or a bottle.

<1-33>

The dicaffeoylquinic acid-containing beverage according to any one of the above-mentioned items <1-1> to <1-32>, in which is subjected to a heat sterilization.

<2-1>

An astringency-suppressing agent for dicaffeoylquinic acids, comprising L-arginine as an active ingredient.

<2-2>

An astringency-suppressing method for dicaffeoylquinic acids, comprising blending L-arginine in a composition containing dicaffeoylquinic acids.

<2-3>

Use of L-arginine for suppressing astringency of dicaffeoylquinic acids.

<2-4>

The astringency-suppressing agent for dicaffeoylguinic acids according to the above-mentioned item <2-1>, the astringency-suppressing method for dicaffeoylquinic acids according to the above-mentioned item <2-2>, or the use of L-arginine according to the above-mentioned item <2-3>, in which the dicaffeoylquinic acids are preferably at least one selected from the group consisting of 3,4-dicaffeoylquinic acid, 3,5-dicaffeoylquinic acid, and 4,5-dicaffeoylquinic acid (the “astringency-suppressing agent for dicaffeoylquinic acids, astringency-suppressing method for dicaffeoylquinic acids, or use of L-arginine” is hereinafter referred to as “astringency-suppressing agent or the like”).

<2-5>

The astringency-suppressing agent or the like according to any one of the above-mentioned items <2-1> to <2-4>, in which the dicaffeoylquinic acids are derived from preferably an extract of one or two or more of plants selected from the group consisting of sunflower seeds, unripe apple, coffee beans, leaves of Simon batatas, cones of Pinaceae plants, seed husks of Pinaceae plants, sugar cane, leaves of Nandina domestics, burdock, the skin of eggplant, the fruit of Japanese apricot, colts foot, and Vitaceae plants, more preferably an extract of coffee beans.

<2-6>

The astringency-suppressing agent or the like according to the above-mentioned item <2-5>, in which the extract of coffee beans is preferably an extract of one or two or more selected from the group consisting of green coffee beans and lightly roasted coffee beans, more preferably an extract of green coffee beans.

<2-7>

The dicaffeoylquinic acid-containing beverage according to the above-mentioned item <2-6>, in which the lightly roasted coffee beans have an L value of preferably 27 or more, more preferably 29 or more, and of preferably less than 62, more preferably 60 or less, even more preferably 55 or less.

<2-8>

The dicaffeoylquinic acid-containing beverage according to the above-mentioned item <2-6> or <2-7>, in which the lightly roasted coffee beans have an L value of preferably 27 or more and less than 62, more preferably from 27 to 60, even more preferably from 29 to 55.

<2-9>

The astringency-suppressing agent or the like according to any one of the above-mentioned items <2-1> to <2-8>, in which the L-arginine is preferably a crystal or crystalline powder.

<2-10>

The astringency-suppressing agent or the like according to any one of the above-mentioned items <2-1> to <2-9>, in which the L-arginine has a purity of 98% or more and a loss on drying of 0.2 mass % or less.

<2-11>

The astringency-suppressing agent or the like according to any one of the above-mentioned items <2-1> to <2-10>, in which is applied to a composition containing dicaffeoylquinic acids.

<2-12>

The astringency-suppressing agent or the like according to any one of the above-mentioned items <2-1> to <2-11>, in which a concentration of the dicaffeoylquinic acids in the composition containing dicaffeoylquinic acids is preferably 0.18 mass % or less, more preferably 0.15 mass % or less, more preferably 0.12 mass % or less, even more preferably 0.10 mass % or less, and is preferably 0.02 mass % or more, more preferably 0.03 mass % or more, more preferably 0.04 mass % or more, even more preferably 0.05 mass % or more.

<2-13>

The astringency-suppressing agent or the like according to any one of the above-mentioned items <2-1> to <2-12>, in which the concentration of the dicaffeoylquinic acids in the composition containing dicaffeoylquinic acids is preferably from 0.02 to 0.18 mass %, more preferably from 0.03 to 0.15 mass %, more preferably from 0.04 to 0.12 mass %, even more preferably from 0.05 to 0.10 mass %.

<2-14>

The astringency-suppressing agent or the like according to any one of the above-mentioned items <2-1> to <2-13>, in which the concentration of the L-arginine in the composition containing dicaffeoylquinic acids is preferably 1.0 mass % or less, more preferably 0.8 mass % or less, more preferably 0.6 mass % or less, more preferably 0.5 mass % or less, more preferably 0.48 mass % or less, even more preferably 0.4 mass % or less, and is preferably 0.1 mass % or more, more preferably 0.12 mass % or more, more preferably 0.14 mass % or more, more preferably 0.16 mass % or more, even more preferably 0.18 mass % or more.

<2-15>

The astringency-suppressing agent or the like according to any one of the above-mentioned items <2-1> to <2-14>, in which the concentration of the L-arginine in the composition containing dicaffeoylquinic acids is preferably from 0.1 to 1.0 mass %, more preferably from 0.12 to 0.8 mass %, more preferably from 0.14 to 0.6 mass %, more preferably from 0.16 to 0.5 mass %, more preferably from 0.18 to 0.48 mass %, even more preferably from 0.18 to 0.4 mass %.

<2-16>

The astringency-suppressing agent or the like according to any one of the above-mentioned items <2-1> to <2-15>, in which a mass ratio between the dicaffeoylquinic acids (A) and the L-arginine (B), [(B)/(A)], is preferably 18 or less, more preferably 17 or less, more preferably 16 or less, more preferably 15 or less, even more preferably 10 or less, and is preferably 2 or more, more preferably 3 or more, more preferably 4 or more, even more preferably 5 or more.

<2-17>

The astringency-suppressing agent or the like according to any one of the above-mentioned items <2-1> to <2-16>, in which the mass ratio between the dicaffeoylquinic acids (A) and the L-arginine (B), [(B)/(A)], is preferably from 2 to 18, more preferably from 3 to 17, more preferably from 4 to 16, more preferably from 5 to 15, even more preferably from 5 to 10.

<2-18>

The astringency-suppressing agent or the like according to any one of the above-mentioned items <2-1> to <2-17>, in which a mass ratio of the dicaffeoylquinic acids (A) to chlorogenic acids is preferably 0.07 or more, more preferably 0.08 or more, more preferably 0.1 or more, even more preferably 0.12 or more, and is preferably 1.0 or less, more preferably 0.9 or less, more preferably 0.8 or less, more preferably 0.6 or less, even more preferably 0.4 or less.

<2-19>

The astringency-suppressing agent or the like according to any one of the above-mentioned items <2-1> to <2-18>, in which the mass ratio of the dicaffeoylquinic acids (A) to chlorogenic acids is preferably 1.0, more preferably from 0.07 to 1.0, more preferably from 0.07 to 0.9, more preferably from 0.08 to 0.8, more preferably from 0.1 to 0.6, even more preferably from 0.12 to 0.4.

<2-20>

The astringency-suppressing agent or the like according to any one of the above-mentioned items <2-1> to <2-19>, in which a concentration of monocaffeoylquinic acids and monoferuloylquinic acids is preferably 0.05 mass % or more, more preferably 0.1 mass % or more, more preferably 0.15 mass % or more, even more preferably 0.2 mass % or more, and is preferably 0. 6 mass % or less, more preferably 0.5 mass % or less, more preferably 0.4 mass % or less, more preferably 0.35 mass % or less, even more preferably 0.3 mass % or less.

<2-21>

The astringency-suppressing agent or the like according to any one of the above-mentioned items <2-1> to <2-20>, in which the concentration of monocaffeoylquinic acids and monoferuloylquinic acids is preferably from 0.05 to 0.6 mass %, more preferably from 0.1 to 0.5 mass %, more preferably from 0.15 to 0.4 mass %, more preferably from 0.2 to 0.35 mass %, even more preferably from 0.2 to 0.3 mass %.

<2-22>

The astringency-suppressing agent or the like according to any one of the above-mentioned items <2-1> to <2-21>, in which the composition containing dicaffeoylquinic acids is preferably an extract of green coffee beans, a coffee beverage, or a concentrated coffee composition.

<2-23>

The astringency-suppressing agent or the like according to the above-mentioned item <2-22>, in which the concentrated coffee composition is preferably a dilutable portion-type beverage or instant coffee.

### EXAMPLES

### Analysis of Chlorogenic Acids

An analysis method for chlorogenic acids was as described below. A HPLC was used as an analyzer.

The model numbers of component units in the analyzer are as follows:
UV-VIS detector: L-2420 (Hitachi High-Technologies Corporation),
Column oven: L-2300 (Hitachi High-Technologies Corporation),
Pump: L-2130 (Hitachi High-Technologies Corporation),
Autosampler: L-2200 (Hitachi High-Technologies Corporation),
Column: Cadenza CD-C18, 4.6 mm (inner diameter)×150 mm (length), particle diameter: 3 μm (Imtakt Corp.).

### Analysis conditions are as follows:

Sample injection volume: 10 μL,
Flow rate: 1.0 mL/min,
Predetermined wavelength of UV-VIS detector: 325 nm,
Predetermined temperature of column oven: 35° C.,
Eluent A: 5 (V/V) % acetonitrile solution containing 0.05 M acetic acid, 0.1 mM 1-hydroxyethane-1,1-diphosphonic acid, and 10 mM sodium acetate,
Eluent B: acetonitrile.

Concentration
gradient conditions

| Time | Eluent A | Eluent B |
|---|---|---|
| 0.0 min. | 100% | 0% |
| 10.0 min. | 100% | 0% |
| 15.0 min. | 95% | 5% |
| 20.0 min. | 95% | 5% |
| 22.0 min. | 92% | 8% |
| 50.0 min. | 92% | 8% |
| 52.0 min. | 10% | 90% |
| 60.0 min. | 10% | 90% |
| 60.1 min. | 100% | 0% |
| 70.0 min. | 100% | 0% |

In HPLC, 1 g of a sample was weighed accurately and diluted with Eluent A in a graduated cylinder to 10 mL total, and the resultant was filtered through a membrane filter (GL chromatodisc 25A, pore diameter: 0.45 μm, GL Sciences Inc.), and was then analyzed. Retention time of chlorogenic acids (unit: minute) Nine of chlorogenic acids
Dicaffeoylquinic acids (diCQA): 3 peaks in total at 36.6, 37.4, and 44.2
Monocaffeoylquinic acids (CQA): 3 peaks in total at 5.3, 8.8, and 11.6
Monoferuloylquinic acids (FQA): 3 peaks in total at 13.0, 19.9, and 21.0

From area values for the nine of chlorogenic acids determined in the foregoing, the content of chlorogenic acids (mass %) was determined by using 5-caffeoylquinic acid as a standard substance.

### Analysis Method for Caffeine

Caffeine was analyzed in the same manner as chlorogenic acids except that the predetermined wavelength of the UV-VIS detector was changed to 270 nm, and caffeine was used as a standard substance. The retention time of caffeine was 18.9 minutes.

### Analysis of Free Arginine

Operating conditions for amino acid automatic analyzer
Type of analyzer: JLC-500/V (JEOL Ltd.)
Column: LCR-6, φ4 mm×120 mm (JEOL Ltd.)
Mobile phase: lithium citrate buffer (P-12 to P-15, P-21) (JEOL Ltd.)
Reaction solution: ninhydrin coloring solution kit-II for JEOL (Wako Pure Chemical Industries, Ltd.)
Flow rate: mobile phase: 0.50 mL/min, reaction solution: 0.30mL/min
Wavelength for measurement: 570 nm

In an autoanalysis of amino acids, 0.5 g of a sample is weighed accurately, and 25 mL of a 10 w/v % sulfosalicylic acid solution is added thereto. In addition, a 3 mol/L sodium hydroxide solution is added thereto, and the whole is mixed. After that, the solution is adjusted to pH 2.2 with sodium citrate buffer and diluted to 100 mL total, and the resultant is filtered through a membrane filter (GL chromatodisc 13A, pore diameter: 0.2 μm, GL Sciences Inc.), and is then analyzed.

### Analysis of Potassium

Measurement was performed using an atomic absorption spectrometer (Hitachi Polarized Zeeman Atomic Absorption Spectrophotometer, type Z-6100).

### Sensory Test

Astringency of the beverages was evaluated by a panel of four members based on the following criteria, and the panel determined final scores based on discussion. The bitterness originating from L-arginine in the beverages of Examples 1 to 11 and the bitterness originating from caffeine in the beverages of Examples 4 and 12 to 16 were evaluated based on the following criteria, and the final scores were determined based on discussion.

Evaluation criteria of astringency
5: Almost no astringency is felt.
4: Astringency is hardly felt.
3: Astringency is slightly felt.
2: Astringency is felt.
1: Astringency is strongly felt.

Evaluation criteria of bitterness originating from L-arginine
A: Almost no bitterness is felt.
B: Bitterness is hardly felt.
C: Bitterness is felt.
D: Bitterness is strongly felt.

Evaluation criteria of bitterness originating from caffeine
5: Almost no bitterness is felt.
4: Bitterness is hardly felt.
3: Bitterness is felt.
2: Slightly strong bitterness is felt. 1: Strong bitterness is felt.

### Production Example 1

A dicaffeoylquinic acid preparation P used in Examples was prepared by the following manner.

Green coffee bean extract powder (solid content: 2.0 g) was dissolved in 20 v/v % aqueous methanol, and the solution was developed by medium-pressure ODS column chromatography (ULTRA PACK ODS-A-40D, 50 mm×300 mm, manufactured by YAMAZEN). The 20 v/v % aqueous methanol was passed through the column at a flow rate of 10 mL/min for 100 minutes, and the concentration of methanol was raised from 20 v/v % to 100 v/v % over 500 minutes to elute chlorogenic acids. Fractionation was performed based on a chromatogram recorded by UV detection at 325 nm. As a result, the fraction Fr. 4 obtained by fractionation at the fourth peak was found to contain dicaffeoylquinic acids. The fraction was freeze-dried, thereby obtaining a dicaffeoylquinic acid preparation P. The preparation P was found to have a dicaffeoylquinic acid content of 75 mass %, a monocaffeoylquinic acid content of 0 mass %, a monoferuloylquinic acid content of 0 mass %, and a caffeine concentration of 0.27 mass %. The potassium content was lower than a detection limit (0.001 mass % or less).

It should be noted that the green coffee bean extract powder was prepared by extraction with hot water at 95° C. from Indonesian green robusta coffee beans, and drying through spray drying.

### Production Example 2

A dicaffeoylquinic acid preparation Q used in Examples was prepared by the following manner.

Green coffee bean extract powder (solid content: 90 g) was dissolved in 360 g of an aqueous ethanol solution containing 60 mass % of ethanol, and the solution was mixed with 45 g of acid clay (MIZUKA ACE #600, manufactured by Mizusawa Industrial Chemicals, Ltd.) and filtered using filter paper precoated with diatomaceous earth. The filtrate was passed through a column filled with 34 mL of coconut shell activated carbon and a column filled with 31 mL of an H-type cation-exchange resin, thereby obtaining a column-treated solution. The column-treated solution was concentrated, thereby obtaining a dicaffeoylquinic acid preparation Q. The preparation Q was found to have a dicaffeoylquinic acid content of 3.5 mass %, a monocaffeoylquinic acid content of 12.9 mass % , a monoferuloylquinic acid content of 2.5 mass %, and a caffeine concentration of 0.0 mass %. The potassium content was 0.34 mass %.

It should be noted that the green coffee bean extract powder was prepared by extraction with hot water at 90° C. from Vietnamese green robusta coffee beans and drying through spray drying.

### Examples 1 to 11 and Comparative Examples 1 to 10

Components were blended at ratios shown in Table 1 to prepare beverages. It should be noted that L-arginine used was a product manufactured by Kyowa Hakko Bio Co., Ltd. The resultant beverages were subjected to an analysis and sensory evaluation. Table 1 shows the results.

TABLE 1

| | | Example | | | | | | |
|---|---|---|---|---|---|---|---|---|
| | | 1 | 2 | 3 | 4 | 5 | 6 | 7 |
| Formulation | diCQA preparation | 0.040 | 0.040 | 0.080 | 0.080 | 0.080 | — | 0.080 |
| of | P [g] | | | | | | | |
| beverage | diCQA preparation | — | — | — | — | — | 1.667 | — |
| | Q [g] | | | | | | | |
| | L-Arginine [g] | 0.120 | 0.480 | 0.180 | 0.390 | 0.480 | 0.480 | 0.540 |
| | Water [g] | Balance | Balance | Balance | Balance | Balance | Balance | Balance |
| | Total [g] | 100.0 | 100.0 | 100.0 | 100.0 | 100.0 | 100.0 | 100.0 |
| Analysis | Concentration of | 0.03 | 0.03 | 0.06 | 0.06 | 0.06 | 0.06 | 0.06 |
| value | diCQA (A) [mass %] | | | | | | | |
| | CQA + FQA [mass %] | 0.00 | 0.00 | 0.00 | 0.00 | 0.00 | 0.26 | 0.00 |
| | Concentration of | 0.12 | 0.48 | 0.18 | 0.39 | 0.48 | 0.48 | 0.54 |
| | L-arginine (B) | | | | | | | |
| | [mass %] | | | | | | | |
| | Concentration of | 0.00011 | 0.00011 | 0.00022 | 0.00022 | 0.00022 | 0.00000 | 0.00022 |
| | caffeine (C) | | | | | | | |
| | [mass %] | | | | | | | |
| | Mass ratio | 4.00 | 16.00 | 3.00 | 6.50 | 8.00 | 8.23 | 9.00 |
| | [(B)/(A)] [—] | | | | | | | |
| | Mass ratio | 1.00 | 1.00 | 1.00 | 1.00 | 1.00 | 0.19 | 1.00 |
| | [(A)/chlorogenic | | | | | | | |
| | acids] [—] | | | | | | | |
| Evaluation | Astringency | 4.0 | 5.0 | 4.0 | 4.5 | 4.5 | 3.5 | 4.5 |
| | Bitterness | A | A | A | A | A | A | B |
| | originating from | | | | | | | |
| | L-arginine | | | | | | | |
| | | Example | | | | Comparative Example | | |
| | | 8 | 9 | 10 | 11 | 1 | 2 | 3 |
| Formulation | diCQA preparation | 0.080 | 0.133 | 0.200 | 0.200 | 0.040 | 0.027 | — |
| of | P [g] | | | | | | | |
| beverage | diCQA preparation | — | — | — | — | — | — | 1.667 |
| | Q [g] | | | | | | | |
| | L-Arginine [g] | 0.960 | 0.480 | 0.480 | 0.975 | — | 0.380 | — |
| | Water [g] | Balance | Balance | Balance | Balance | Balance | Balance | Balance |
| | Total [g] | 100.0 | 100.0 | 100.0 | 100.0 | 100.0 | 100.0 | 100.0 |
| Analysis | Concentration of | 0.06 | 0.10 | 0.15 | 0.15 | 0.03 | 0.02 | 0.06 |
| value | diCQA (A) [mass %] | | | | | | | |
| | CQA + FQA [mass %] | 0.00 | 0.00 | 0.00 | 0.00 | 0.00 | 0.00 | 0.26 |
| | Concentration of | 0.96 | 0.48 | 0.48 | 0.98 | 0.00 | 0.38 | 0.00 |
| | L-arginine (B) | | | | | | | |
| | [mass %] | | | | | | | |
| | Concentration of | 0.00022 | 0.00036 | 0.00054 | 0.00054 | 0.00011 | 0.00007 | 0.00000 |
| | caffeine (C) | | | | | | | |
| | [mass %] | | | | | | | |
| | Mass ratio | 16.00 | 4.80 | 3.20 | 6.50 | 0.00 | 19.00 | 0.00 |
| | [(B)/(A)] [—] | | | | | | | |
| | Mass ratio | 1.00 | 1.00 | 1.00 | 1.00 | 1.00 | 1.00 | 0.19 |
| | [(A)/chlorogenic | | | | | | | |
| | acids] [—] | | | | | | | |
| Evaluation | Astringency | 3.5 | 4.0 | 4.0 | 3.0 | 1.0 | 5.0 | 1.0 |
| | Bitterness | B | A | A | B | A | C | A |
| | originating from | | | | | | | |
| | L-arginine | | | | | | | |
| | | Comparative Example | | | | | | |
| | | 4 | 5 | 6 | 7 | 8 | 9 | 10 |
| Formulation | diCQA preparation | 0.080 | 0.080 | 0.133 | 0.200 | 0.000 | 0.033 | 0.427 |
| of | P [g] | | | | | | | |
| beverage | diCQA preparation | — | — | — | — | — | — | — |
| | Q [g] | | | | | | | |
| | L-Arginine [g] | 0.090 | 1.140 | 0.150 | 1.050 | 0.480 | 0.475 | 0.480 |
| | Water [g] | Balance | Balance | Balance | Balance | Balance | Balance | Balance |
| | Total [g] | 100.0 | 100.0 | 100.0 | 100.0 | 100.0 | 100.0 | 100.0 |
| Analysis | Concentration of | 0.06 | 0.060 | 0.10 | 0.15 | 0.00 | 0.025 | 0.32 |
| value | diCQA (A) [mass %] | | | | | | | |
| | CQA + FQA [mass %] | 0.00 | 0.000 | 0.00 | 0.00 | 0.00 | 0.000 | 0.00 |
| | Concentration of | 0.09 | 1.14 | 0.15 | 1.05 | 0.48 | 0.48 | 0.48 |
| | L-arginine (B) | | | | | | | |
| | [mass %] | | | | | | | |
| | Concentration of | 0.00022 | 0.00022 | 0.00036 | 0.00054 | 0.00000 | 0.00009 | 0.00115 |
| | caffeine (C) | | | | | | | |
| | [mass %] | | | | | | | |
| | Mass ratio | 1.50 | 19.00 | 1.50 | 7.00 | — | 19.00 | 1.50 |
| | [(B)/(A)] [—] | | | | | | | |
| | Mass ratio | 1.00 | 1.00 | 1.00 | 1.00 | 1.00 | 1.00 | 1.00 |
| | [(A)/chlorogenic | | | | | | | |
| | acids] [—] | | | | | | | |
| Evaluation | Astringency | 2.0 | 3.5 | 1.5 | 3.0 | 5.0 | 5.0 | 1.0 |
| | Bitterness | A | D | A | C | C | C | B |
| | originating from | | | | | | | |
| | L-arginine | | | | | | | |

diCQA: dicaffeoylquinic acids
diCQA preparation P: dicaffeoylquinic acid preparation obtained in Production Example 1
diCQA preparation Q: dicaffeoylquinic acid preparation obtained in Production Example 2
CQA + FQA: total concentration of monocaffeoylquinic acids and monoferuloylquinic acids
Mass ratio [(A)/chlorogenic acids]: dicaffeoylquinic acids (A)/chlorogenic acids

Table 1 shows that the dicaffeoylguinic acid-containing beverage containing a high concentration of dicaffeoylquinic acids and having suppressed astringency originating from dicaffeoylquinic acids can be obtained by controlling the concentration of the dicaffeoylquinic acids (A), the concentration of the L-arginine (B) , and the content mass ratio between the dicaffeoylquinic acids (A) and the L-arginine (B) in the beverage within specific ranges.

### Examples 12 to 16

Components were blended at ratios shown in Table 2 to prepare beverages. It should be noted that L-arginine used was a product manufactured by Kyowa Hakko Bio Co., Ltd. The resultant beverages were subjected to an analysis and sensory evaluation. Table 2 shows the results together with the results of Example 4.

TABLE 2

| | | Example | | | | | |
|---|---|---|---|---|---|---|---|
| | | 4 | 12 | 13 | 14 | 15 | 16 |
| Formulation | diCQA preparation P [g] | 0.080 | 0.080 | 0.080 | 0.080 | 0.080 | 0.080 |
| of beverage | L-Arginine [g] | 0.390 | 0.390 | 0.390 | 0.390 | 0.390 | 0.390 |
| | Anhydrous caffeine [g] | 0.0000 | 0.0013 | 0.0028 | 0.0048 | 0.0098 | 0.0198 |
| | Water [g] | Balance | Balance | Balance | Balance | Balance | Balance |
| | Total [g] | 100.0 | 100.0 | 100.0 | 100.0 | 100.0 | 100.0 |
| Analysis value | Concentration of diCQA (A) [mass %)] | 0.06 | 0.06 | 0.06 | 0.06 | 0.06 | 0.06 |
| | CQA + FQA [mass %] | 0.00 | 0.00 | 0.00 | 0.00 | 0.00 | 0.00 |
| | Concentration of L-arginine (B) | 0.39 | 0.39 | 0.39 | 0.39 | 0.39 | 0.39 |
| | [mass %] | | | | | | |
| | Concentration of caffeine (C) | 0.00022 | 0.00152 | 0.00302 | 0.00502 | 0.01002 | 0.02002 |
| | [mass %] | | | | | | |
| | Mass ratio [(B)/(A)] [—] | 6.50 | 6.50 | 6.50 | 6.50 | 6.50 | 6.50 |
| | Mass ratio [(A)/chlorogenic acids] | 1.00 | 1.00 | 1.00 | 1.00 | 1.00 | 1.00 |
| | [—] | | | | | | |
| Evaluation | Astringency | 4.5 | 4.5 | 4.5 | 4.5 | 4.5 | 4.5 |
| | Bitterness originating from | 5.0 | 5.0 | 4.5 | 4.5 | 4.0 | 3.5 |
| | caffeine | | | | | | |

diCQA: dicaffeoylquinic acids
diCQA preparation P: dicaffeoylquinic acid preparation obtained in Production Example 1
CQA + FQA: total concentration of monocaffeoylquinic acids and monoferuloylquinic acids
Mass ratio [(A)/chlorogenic acids]: dicaffeoylquinic acids (A)/chlorogenic acids

Table 2 shows that when the beverage contains caffeine, the dicaffeoylquinic acid-containing beverage having suppressed bitterness originating from caffeine can be obtained as long as the concentration of the caffeine (C) is 0.02 mass % or less.

1-12. (canceled)

13. A dicaffeoylquinic acid-containing beverage, comprising components (A) and (B):
(A) 0.02 to 0.18 mass % of dicaffeoylquinic acids; and
(B) 0.1 to 0.48 mass % of L-arginine,
wherein a mass ratio between component (A) and component (B), [(B)/(A)], is from 2 to 18.

14. The dicaffeoylquinic acid-containing beverage according to claim 13, wherein the component (A) is derived from an extract of at least one member selected from the group consisting of green coffee beans and lightly roasted coffee beans.

15. The dicaffeoylquinic acid-containing beverage according to claim 14, wherein the lightly roasted coffee beans have an L-value of 27 or more and less than 62.

16. The dicaffeoylquinic acid-containing beverage according to claim 13, further comprising at least one member selected from the group consisting of monocaffeoylquinic acids and monoferuloylquinic acids.

17. The dicaffeoylquinic acid-containing beverage according to claim 13, wherein a mass ratio between the dicaffeoylquinic acids (A) and chlorogenic acids, is from 0.07 to 1.0.

18. The dicaffeoylquinic acid-containing beverage according to claim 13, wherein a content of the component (B) is from 0.18 to 0.48 mass %.

19. The dicaffeoylquinic acid-containing beverage according to claim 13, further comprising less than 0.003 mass % of caffeine (C).

20. An astringency-suppressing method for dicaffeoylquinic acids, comprising blending L-arginine in a composition containing dicaffeoylquinic acids.

21. The astringency-suppressing method according to claim 20, wherein a concentration of the dicaffeoylquinic acids in the composition is from 0.02 to 0.18 mass %.

22. The astringency-suppressing method according to claim 20, wherein the L-arginine is blended so that a concentration of the L-arginine in the composition is from 0.1 to 1.0 mass %.

23. The astringency-suppressing method according to claim 20, wherein a mass ratio between the L-arginine (B) and the dicaffeoylquinic acids (A) in the composition, [(B)/(A)], is adjusted to from 2 to 18.

24. The astringency-suppressing method according to claim 21, wherein the L-arginine is blended so that a concentration of the L-arginine in the composition is from 0.1 to 1.0 mass %.

25. The dicaffeoylquinic acid-containing beverage according to claim 13, wherein a content of potassium (D) is from 0.00001 to 0.06 mass %.

26. The dicaffeoylquinic acid-containing beverage according to claim 25, wherein a content mass ratio between the dicaffeoylquinic acids (A) and the potassium (D), [(D)/(A)], is from 0.0005 to 0.5.

27. The dicaffeoylquinic acid-containing beverage according to claim 14, wherein a mass ratio of the dicaffeoylquinic acids (A) to chlorogenic acids, is from 0.07 to 1.0.

28. The dicaffeoylquinic acid-containing beverage according to claim 14, further comprising less than 0.003 mass % of caffeine (C).

29. The dicaffeoylquinic acid-containing beverage according to claim 27, further comprising less than 0.003 mass % of caffeine (C).

30. The dicaffeoylquinic acid-containing beverage according to claim 29, wherein a content of potassium (D) is from 0.00001 to 0.06 mass %.

31. The dicaffeoylquinic acid-containing beverage according to claim 30, wherein a content mass ratio between the dicaffeoylquinic acids (A) and the potassium (D), [(D)/(A)], is from 0.0005 to 0.5.
